(19) United States
(12) Patent Application Publication
(10) Pub. No.: US 2008/0269311 A1
(43) Pub. Date: Oct. 30, 2008

(54) Combination of Dpp-Iv Inhibitors and Compounds Modulating 5-Ht3 and/or 5-Ht4 Receptors

(76) Inventor: Edwin Bernard Villhauer, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/572,060

(22) PCT Filed: Jul. 13, 2005
(86) PCT No.: PCT/EP05/07636

§ 371 Date: Jan. 11, 2008

Related U.S. Application Data
(60) Provisional application No. 60/588,011, filed on Jul. 14, 2004.

(51) Int. Cl. A61K 31/404; A61K 31/40; A61P 3/00

(52) U.S. Cl. 514/418; 514/423

(57) ABSTRACT

The present invention relates to a combination, such as a combined preparation or pharmaceutical composition, respectively, comprising of a DPP IV inhibitor or a pharmaceutically acceptable salt thereof and comprising at least one therapeutic agent selected from an agent interacting with a 5-HT₃ receptor and/or an agent interacting with 5-HT₄ receptor, or a pharmaceutically acceptable salt thereof.

Serotonin has been known for some years to modulate peristalsis in the gastro-intestinal (GI) tract in various mammalian models. During the mid 1980s, several specific antagonists to the 5-HT₃ receptor subtype were identified and are currently used as anti-emesis/vomiting agents in cancer therapy. 5-HT₃ antagonists have also recently been studied for the treatment of irritable bowel syndrome (“IBS”).

A number of gastrointestinal syndromes are related to the production and actions of serotonin, and they have a fairly common occurrence in a very large number of people worldwide. Some of the more well-known gastrointestinal conditions, syndromes or diseases are IBS, gastro-esophageal reflux disease (“GERD”) and dyspepsia.

IBS is a chronic condition associated with abdominal pain, bloating and altered bowel function and is estimated to affect as much as 10-20% of the population. Sometimes the disease is referred to as irritable colon, spastic colon, spastic colitis or mucous colitis. The latter two are almost certainly misnomers, as colitis implies inflammation of the colon, and an absence of inflammation is one of the defining observations in a diagnosis of IBS. The cause of IBS is unknown, but a number of factors have been implicated, including diet, lifestyle, depression, anxiety, infections and unrelated inflammatory conditions, including early insult resulting in central neuronal sensitization and sensitizing of neurons in the gut. Almost all medications currently in use for the treatment of IBS have failed to establish a significant therapeutic effect.

GERD is a condition that is associated with the reflux of gastric contents to the esophagus through the lower esophageal sphincter. GERD is characterized by symptoms of heartburn, bloating, abdominal pain, epigastric pain, early satiety, nausea, regurgitation, burbulence and vomiting. The reflux is thought to occur because of an increased incidence of transient lower esophageal sphincter relaxations allowing gastric contents to enter the esophagus.

Dyspepsia is also an important health problem. The most common conditions that are associated with patients who present with chronic symptoms of dyspepsia are GERD, duodenal ulcer or gastric ulcer and other diagnoses (e.g. functional/non-ulcerative dyspepsia, gallbladder or liver disease).

These conditions or diseases are characterized by altered motility, sensitivity, secretion and/or infections with Helicobacter pylori as well as potentially a psychological (usually subconscious) overlay. At present, only few medications have shown clinically significant efficacy for the treatment of e.g. functional dyspepsia, of which some do exert various adverse effects in man.

GLP-1 derivatives where also described in the patent application US 2003216292 as potentially effective for treating gastrointestinal disorders.

Accordingly, there is a need for agents or treatments which modulate and normalize altered GI motility, sensitivity and secretion, and which have broad clinical usefulness for the treatment of the numerous gastrointestinal disorders affecting millions of people each year.

It has now been found that a combination comprising at least one agent interacting with a 5-HT₃ or a 5-HT₄ receptor, e.g., as defined below, and a DPP-IV inhibitor as co-agent, e.g., as defined below, has a beneficial effect and is useful in the treatment of altered gastrointestinal motility, sensitivity and/or secretion and/or abdominal disorders and conditions/disorders that might be treated by DPP-IV inhibition. This combination may also be used to regulate, stabilize and normalize altered gastrointestinal motility, sensitivity and/or secretion and/or abdominal disorders.

Thus, the present invention relates to combinations comprising
i) a DPP IV inhibitor or a pharmaceutically acceptable salt thereof, and
ii) at least one therapeutic agent selected from an agent interacting with a 5-HT₃ and/or an agent interacting with 5-HT₄ receptor, or a pharmaceutically acceptable salt thereof.

Preferably the present invention relates to a combination, such as a combined preparation or pharmaceutical composition, respectively, comprising a DPP IV inhibitor or a pharmaceutically acceptable salt thereof, and at least one therapeutic agent selected from the group comprising

a) an agent interacting with a 5-HT₃ receptor or a pharmaceutically acceptable salt thereof,
b) an agent interacting with a 5-HT₄ receptor or a pharmaceutically acceptable salt thereof, and at least one additional pharmaceutically acceptable carrier.

Preferably the combination is a pharmaceutical composition or a combined pharmaceutical preparation.

In this pharmaceutical composition, the combination partners (i) and (ii) can be administered together, one after the other or separately in one combined unit dosage form or in two separate unit dosage forms. The unit dosage form may also be a fixed combination.

The term “at least one therapeutic agent” shall mean that in addition to the DPP IV inhibitor one or more, for example two, furthermore three, active ingredients as specified according to the present invention can be combined. Preferably a combination partner a) or b) or one combination partner selected from a) and one selected from b).

The term “DPP-IV” as used herein is intended to mean dipeptidyl peptidase IV, also known as CD26. DPP-IV, a serine protease belonging to the group of post-proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% or 20-80% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998,DE19616 486 A1, WO 00/34241, WO 95/15309, WO 01/72290, WO01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14,15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857 and WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables 1a to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890.

WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60, U.S. application Ser. No. 09/788,173 filed Feb. 16, 2001 (attorney file LA50) especially the described examples, WO99/38501 especially the described examples, WO99/46272 especially the described examples and DE1 9616 486 A1 especially val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. Nos. 6,124,305 and U.S. Pat. No. 6,107,317, International Patent Applications, Publication Numbers WO 95153 09 and WO 9818763.

In each case in particular in the compound claims and the final products of the working examples, the subject matter of the final products, the pharmaceutical preparations and the claims are hereby incorporated into the present application by reference to these publications.

Published patent application WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728).

DE19616 486 A1 discloses val-pyr, val-thiazolididc, isoleucyl-thiazolidide, isoleucyl-pyrrolidide, and fumar salts of isoleucyl-thiazolidide and isoleucyl-pyrrolidide.

Published patent application WO 0034241 and published patent US 6110949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4. In particular these applications describe the compound 1-[[(3-Hydroxy-1-adamantyl)amino]acetyl]-2-cyano-(S)-pyrrolidine (also known as LAF237 or vildagliptin).

Published patent application WO 9515309 discloses amino acid 2-cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20.

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV. Other preferred DPP-IV inhibitors are the compounds of formula I, II or III disclosed in the patent application WO 03/057200 on page 14 to 27. Most preferred DPP-IV inhibitors are the compounds specifically described on pages 28 and 29. Any of the substances disclosed in the above mentioned patent documents, hereby included by reference, are considered potentially useful as DPP-IV inhibitors to be used in carrying out the present invention.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII

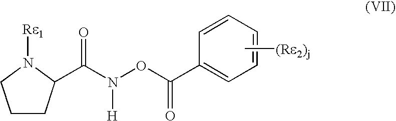

wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIa

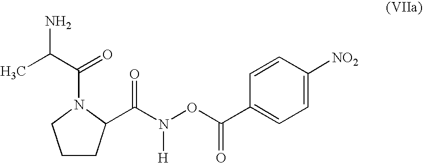

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (IS,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

FE-999011 is described in the patent application WO 95/15309 page 14, as compound No. 18.

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

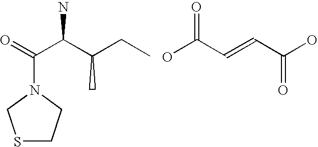

and is described in WO 99/61431 and also in Diabetes 1998, 47, 1253-1258, in the name of Probiodrug, as well as the compound P93101 described by the same company.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula:

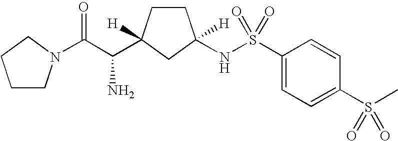

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

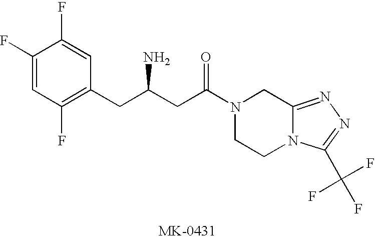

described by the example 7 and also known as MK-0431 or Sitagliptin.

Preferred DPP-IV inhibitors are also described in the patent application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

[S]-1-[2-(5-cyano-2-pyridinylamino)ethylamino]acetyl-2-pyrrolidine carbonitrile monohydrochloride and (S)-1-[(3-hydroxy-1-adamantyl)amino]acetyl-2-cyano-pyrrolidine are specifically disclosed in Example 3 of WO 98/19998 and Example 1 of WO 00/34241, respectively. The DPP-IV inhibitor P32/98 (see above) is specifically described in Diabetes 1998, 47, 1253-1258. [S]-1-[2-(5-cyano-2-pyridinylamino)ethylamino]acetyl-2-pyrrolidine carbonitrile monohydrochloride and (S)-1-[(3-hydroxy-1-adamantyl)amino]acetyl-2-cyano-pyrrolidine can be formulated as described on page 20 of WO 98/19998 or in WO 00/34241.

Especially preferred are 1-{2-[(5-cyanopyridin-2-yl)amino]ethylamino}acetyl-2 (S)-cyano-pyrrolidine dihydrochloride (DPP728) (also named [S]-1-[2-(5-cyano-2-pyridinylamino)ethylamino]acetyl-2-pyrrolidine carbonitrile monohydrochloride), of formula

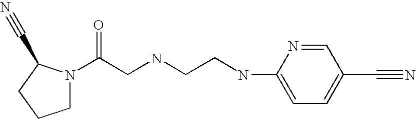

especially the dihydrochloride and monohydrochloride thereof,
and 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin) of formula

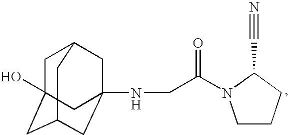

and L-threo-isoleucyl thiazolidine (compound code according to Probiodrug: P32/98 as described above), MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

DPP728 and LAF237 are specifically disclosed in Example 3 of WO 98/19998 and Example 1 of WO 00/34241, respectively. The DPP-IV inhibitor P32198 (see above) is specifically described in Diabetes 1998, 47, 1253-1258. DPP728 and LAF237 can be formulated as described on page 20 of WO 98/19998 or in WO 00/34241 or in the International Patent Application No. EP2005/000400 (application number).

Especially preferred are orally active DPP-IV inhibitors.

Any of the substances disclosed in the above mentioned patent documents or scientific publications, hereby included by reference, are considered potentially useful as DPP-IV inhibitors to be used in carrying out the present invention.

In each case in particular in the compound claims and the final products of the working examples, the subject matter of the final products, the pharmaceutical preparations and the claims are hereby incorporated into the present application by reference to these publications.

Agent interacting with a 5-HT₄ receptor include, 5-HT₄ receptor partial agonists, 5-HT₄ receptor agonists, 5-HT₄ receptor antagonists or dual 5HT3 and 5HT4 agonists.

Representative 5-HT₄ receptor partial agonists include, but are not limited to, compounds as described in the U.S. Pat. No. 5,510,353 especially the examples 1 to 117, having an intrinsic activity less than that of serotonin (The corresponding subject matter of this reference is herewith incorporated by reference in this specification).

Preferred compounds as 5-HT₄ receptor partial agonists are e.g. those described in U.S. Pat. No. 5,510,353 wherein R₁ is H, Z is —CH═ and R₅ is OH or C₁₋₆alkoxy.

Further examples of 5-HT₄ receptor partial agonists include e.g. RS 67333 (1-(4-amino-5-chloro-2-methoxyphenyl)-3-[1-butyl-4-piperidinyl]-1-propanone), or RS 67506 (1-(4-amino-5-chloro-2-methoxyphenyl)-3-[1-methylsulphonylamino)ethyl-4-piperidinyl]-1-propanone).

A particularly preferred compound described in U.S. Pat. No. 5,510,353 is the compound of formula

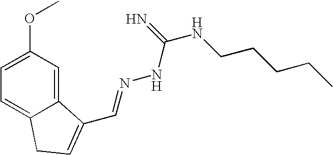

in free form or in pharmaceutically acceptable salt form. This compound has the chemical name of 3-(5-methoxy-1H-indol-3-yl-methylene)-N-pentylcarbazimidamide, and is also known as tegaserod and under the trade marks ZELMAC and ZELNORM. It is described in the U.S. Pat. No. 5,510,353 and European patent no. 505322 as example 13, and disclosed as being a 5-HT₄ receptor partial agonist. It may also exist in form of tautomers

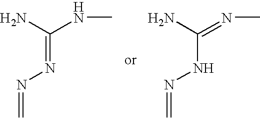

which are included in the present invention. A preferred salt form is the hydrogen maleate.

The 5-HT₄ receptor agonists as a co-agent in combination include any compound which can activate 5-HT₄ receptors under quiescent/resting conditions. These compounds include, but are not limited to, compounds of formula I as disclosed in EP-B1-0 505 322, cisapride, nor-cisapride, renzapride, zacopride, mosapride, prucalopride, buspirone, norcisapride; 4-amino-5-chloro-2-methoxy-N-(1-substituted piperidin-4-yl)benzamide known as Y-34959; SB 205149, SC 53116, RS 67333, RS 67506, BIMU-1, BIMU-8 and (S)-RS 56532, compounds described in the US patent application No. 20040127514 especially examples 1 to 22, or the US patent application No. 20040122043 especially examples 1 to 9, or the US patent application No. US20040034226 especially examples 1 to 30, or the U.S. Pat. No. 6,624,162 especially examples 1 to 30. A variety of imidazopyridine 5-HT 4 receptor modulators compounds were disclosed in U.S. Application No. 60/343,371, filed on Oct. 22, 2001. Cisapride, cis-4-amino-5-chloro-N-[1-[3-(4-fluorphenoxy)propyl]-3-methoxy-4-piperidinyl]-2-methoxy-benzamide, is in use as a gastro-prokinetic agent (See A. Reyntjens et al., Drug Div. Res., 8, 251 (1986) and Curr. Ther. Res., 36, 1029-1070 (1984)). The compound is marketed internationally under trade names such as ACENALIN®, PREPULSID®, RISAMOL®, PULSAR® and PROPULSIN®.

A preferred group of 5-HT₄ receptor agonists or partial agonists are those which are selective; by selective is meant a compound which does not substantially bind to or stimulate the 5-HT₃ receptor subtype. Tegaserod, for example, does neither bind to nor stimulate the 5-HT₃ receptor subtype.

5-HT₄ receptor antagonists for use as a co-agent in combination 1.1 or 1.2 or as a first agent in combination 1.3 or 1.4 include any compounds which bind to the 5-HT₄ receptor as defined by the IUPHAR (Pharmacological Reviews, Vol. 44, p. 157-213, 1994) and that do not activate the 5-HT₄ receptor and antagonize the effects of serotonin. A relevant test to determine whether or not a compound is a 5-HT₄ receptor antagonist is the Guinea-Pig distal colon test as described in Br. J. Pharm., p. 1593-1599 (1993) or in the test described in Arch. Pharmacol., Vol. 343, p. 439-446 (1991). Representative 5-HT₄ receptor antagonists include e.g. piboserod; A-85380 (Abbott Laboratories) (WO 94/08994); SB 204070 (SmithKline Beecham) (Drugs Fut., 19:1109-1121, 1994); SB 207058 (Exp. Opin. Invest. Drugs, 3(7):767, 1994); SB 207710 (Drug Data Report, 15(10):949, 1993); SB 205800 (Drug Data Report, 15(10):949, 1993); SB 203186 (Br. J. Pharmacol., 110:1023-1030, 1993); N 3389 (Nisshin Flour Milling) (Eur. J. Pharmacol., 271:159, 1994); FK 1052 (Fujisawa) (J. Pharmacol. Exp. Ther., 265:752, 1993); SC 56184 (Searle) (R&D Focus, 2(37) 10, 1993); SC 53606 (Searle/Monsanto) (J. Pharmacol. Exp. Ther. 226:1339, 1993); DAU 6285 (Boerhinger Ingelheim) (Br. J. Pharmacol., 105:973, 1992); GR 125487 (Glaxo) (Br. J. Pharmacol., 13 suppl. 119P & 120P, 1994); GR 113808 (Br. J. Pharmacol. 110:1172, 1993); RS 23597 (Syntex) (Bioorg Med. Chem. Lett., 4(20):2477, 1994); RS 39604 (Br. J. Pharmacol., 115, 1087-1095, 1995); LY0353433 (Eli Lilly Co. Ltd.) (J. Pharmacol. Exp. Ther., 277(1), 97-104,1996); and R59595 (Eur. J. Pharmacol., 212, 51-59,1992). 5-HT₄ receptor antagonist piboserod or SB-207266A have also been suggested for the treatment of IBS.

Dual 5HT3 and 5HT4 agonists include renzapride (SmithKline Beecham) and E3620 (Eisai). A 5HT1a agonist is also known, LY315535 (Eli Lilly).

Agent interacting with a 5-HT₃ receptor include 5-HT₃ receptor antagonists and Dual 5HT3 and 5HT4 agonists.

5-HT₃ receptor antagonists include, e.g. cilansetron which is described in EP 29761; alosetron which is described in WO 99/17755; ramosetron; azasetron; ondansetron; dolasetron; ramosetron; granisetron; mirtazapine; indisetron; lerisetron; Ro-93777; YM-114; talipexole; N-3389, zacopride, cilansetron, E-3620, lintopride, KAE-393, itasetron, mosapride; dolasetron and tropisetron, or compounds described in US patent application No. US20030158221 especially examples 1 to 30.

In UK Patent No. 2209335 there is disclosed, inter alia, the compound 2,3,4,5-tetrahydro-5-methyl-2-[(5-methyl-1H-imidazol-4-yl)methyl]-1H-pyrido[4,3-b]indol-1-one, now known as alosetron, and pharmaceutically acceptable salts, solvates and pharmaceutically acceptable equivalents thereof, in particular its hydrochloride salt.

Compounds which show characteristics of 5-HT₃ receptor antagonists and 5-HT₄ receptor agonists or antagonists for use as a co-agent in combination 1.1 or 1.2 or as first agent in combination 1.3 are e.g. cisapride and nor-cisapride; BIMU compounds, for example BIMU1, BIMU8 and DAU 6215 (also known as itasetron) as disclosed in Dumuis A., et al., Naunyn Schmiedeber's Arch. Pharmacol., Vol. 343(3), pp. 245-251 (1991); DAU-6236 as disclosed in Rizzi, C. A. et al., J. Pharmacol. Exp. Ther., Vol. 261, pp. 412-419 (1992); and DAU-6258, Turconi M, et al., J. Med. Chem., Vol. 33(8), pg. 2101-2108 (1990), SDZ 205-557 which is a benzoic acid derivative (ester) Eglen R. M. et al., Proc. Br. Pharmacol. Soc., Vol. 149 (1992); renzapride; zacopride; SB 205149; SC 53116; RS 67333; RS 67506; or (S)-RS 56532, lintopride.

The dosage of the agent interacting with a 5-HT₃ receptor or the agent interacting with a 5-HT₄ receptor, administered will also be generally dependent upon the health of the subject being treated, the extent of gastro-intestinal treatment desired, the nature and kind of concurrent therapy, if any, and the frequency of treatment and nature of the effect desired. In general, the dosage of the agent is generally in the range of from about 0.001 to about 50 mg/kg body weight of the subject per day, preferably from about 0.1 to about 10 mg/kg body weight of the subject per day, administered as a single or divided dose. However, some variability in the general dosage range may also be required depending upon the age, weight, and species of the patient, the intended route of administration, and the progress and degree of severity of the disease or condition being treated.

Daily dosages of the agent interacting with a 5-HT₃ receptor or the agent interacting with a 5-HT₄ receptor required in practicing the method of the present invention will vary depending upon, for example the mode of administration and the severity of the condition to be treated. An indicated daily dose is in the range of from about 1 to about 200 mg, e.g. from 2 to 30 mg or from 2 to 24 mg or from 2 to 12 mg, of active agent for oral use, conveniently administered once or in divided dosages.

Preferred are combinations, such as combined preparations or pharmaceutical compositions, respectively, comprising a DPP-IV inhibitor preferably LAF237 or a pharmaceutically accepted salt thereof and as second active agent an active agent selected from the group consisting of tegaserod, cisapride, nor-cisapride, renzapride, zacopride, mosapride, prucalopride, buspirone, and norcisapride or, in each case, a pharmaceutically acceptable salt thereof, especially tegaserod hydrogen maleate.

Furthermore preferred are combinations, such as a combined preparations or pharmaceutical compositions, respectively, comprising a DPP-IV inhibitor preferably LAF237 or a pharmaceutically accepted salt thereof and one active agent selected from the group consisting of cilansetron, ramosetron, azasetron, ondansetron, dolasetron, ramosetron, granisetron, mirtazapine, indisetron, lerisetron, Ro-93777, YM-114, talipexole, N-3389, zacopride, cilansetron, E-3620, lintopride, KAE-393, itasetron, mosapride; dolasetron and tropisetron or, in each case, a pharmaceutically acceptable salt thereof.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a DPP IV inhibitor or a salt thereof and at least one therapeutic agent selected from (i) to (ii) results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the DPP-IV inhibitor with at least one therapeutic agent selected from (i) to (ii) results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on irritable bowel syndrome, gastro-esophageal reflux disease, dyspepsia, diabetes especially type II diabetes, IGT and diseases and conditions associated with diabetes mellitus, IGT, obesity, for a number of combinations as described herein.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these references is herewith incorporated by reference in this specification.

Accordingly, the combination according to the present invention may be used, e.g., for the prevention, delay of progression or treatment of diseases and disorders that may be inhibited by DPP IV inhibition and/or by interacting with a 5-HT₃ or a 5-HT₄ receptors.

Thus in a further aspect the present invention concerns the use of a combination comprising
i) a DPP IV inhibitor or a pharmaceutically acceptable salt thereof, and
ii) at least one therapeutic agent selected from an agent interacting with a 5-HT₃ receptor and/or an agent interacting with a 5-HT₄ receptor, or a pharmaceutically acceptable salt thereof
   for the manufacture of a medicament for the prevention, delay of progression or treatment of diseases and disorders that may be inhibited by DPP IV inhibition and/or by interacting with a 5-HT₃ receptor or a 5-HT₄ receptors.

The invention furthermore relates to a method for the prevention of, delay of progression of, treatment of diseases and disorders that may be inhibited by DPP IV inhibition and/or by interacting with a 5-HT₃ receptor or a 5-HT₄ receptors,

comprising administering to a warm-blooded animal, including man, in need thereof a jointly effective amount of a combination of a DPP IV inhibitor or a pharmaceutically acceptable salt thereof with at least one therapeutic agent selected from an agent interacting with a 5-HT₃ receptor and/or an agent interacting with a 5-HT₄ receptor, or a pharmaceutically acceptable salt thereof;
and at least one additional pharmaceutically acceptable carrier.

The invention furthermore relates to a pharmaceutical composition for the prevention of, delay of progression of, treatment of a disease or condition selected from diseases and disorders that may be inhibited by DPP IV inhibition and/or by interacting with a 5-HT₃ receptor or a 5-HT₄ receptors, comprising a combination of a DPP IV inhibitor or a pharmaceutically acceptable salt thereof with at least one therapeutic agent selected from an agent interacting with a 5-HT₃ receptor and/or an agent interacting with a 5-HT₄ receptor, or a pharmaceutically acceptable salt thereof;

and at least one additional pharmaceutically acceptable carrier.

In an additional embodiment, the present invention concerns;
1. the use of a DPP IV inhibitor or a pharmaceutically acceptable salt thereof, for the manufacture of a medicament for the prevention, delay of progression or treatment of diseases and disorders selected from altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, e.g. diabetic gastroparesis, ulcerative colitis, Crohn's disease, ulcers and the visceral pain associated therewith.
2. a method for the prevention of, delay of progression of, treatment of diseases and disorders that may be inhibited by DPP IV inhibition and/or by interacting with a 5-HT₃ receptor or a 5-HT₄ receptors, comprising administering to a warm-blooded animal, including man, in need thereof an effective amount of a DPP IV inhibitor or a pharmaceutically acceptable salt thereof and at least one additional pharmaceutically acceptable carrier.
3. A method for the prevention of, delay of progression of, treatment of diseases and disorders selected from altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, e.g. diabetic gastroparesis, ulcerative colitis, Crohn's disease, ulcers and the visceral pain associated therewith, comprising administering to a warm-blooded animal, including man, in need thereof an effective amount of a DPP IV inhibitor or a pharmaceutically acceptable salt thereof.

Methods or uses as described above, wherein the disease or condition is selected from insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, e.g. diabetic gastroparesis, ulcerative colitis, Crohn's disease, ulcers and the visceral pain associated therewith.

Most preferably, the disease or condition is selected from diabetes, type 2 diabetes, IGT and diseases or conditions associated with diabetes.

Most preferably, the disease or condition is selected from irritable bowel syndrome (IBS), gastro-esophageal reflux disease (GERD), diabetic gastropathy, diabetic gastroparesis, chronic constipation and dyspepsia.

Preferred combinations for the described uses or methods are described herein.

A “disease or condition which may be inhibited by a DPP-IV inhibitor” as defined in this application comprises, but is not limited to insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance. Preferably, a “disease or condition which may be inhibited by a DPP-IV inhibitor” is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy and foot ulcerations.

The term “curative” as used herein means efficacy in treating ongoing diseases, disorder or conditions.

The term “prophylactic” means the prevention of the onset or recurrence of diseases, disorders or conditions to be treated.

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

The term “combined pharmaceutical preparation” as that term is used herein means that the active ingredients, e.g. tegaserod or tegaserod hydrogen maleate and a DPP-IV inhibitor preferably LAF237, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

A “disease or condition which may be inhibited by interacting with a 5-HT₃ or a 5-HT₄ receptors” is preferably an altered gastrointestinal motility, sensitivity and/or secretion disorder(s).

In the present description, the term “treatment” includes both prophylactic or preventative treatment as well as curative or disease suppressive treatment, including treatment of patients at risk of contracting the disease or suspected to have contracted the disease or disorder as well as ill patients. This term further includes the treatment for the delay of progression of the disease.

The term “altered gastrointestinal motility, sensitivity and/or secretion disorder(s)” as used herein includes one or more of the symptoms and conditions which affect the gastrointestinal tract from the mouth to the anus, which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, e.g. diabetic gastroparesis, ulcerative colitis, Crohn's disease, ulcers and the visceral pain associated therewith.

The term “abdominal disorder(s)” as used herein includes those conditions which affect the lower abdomen and include but are not limited to those conditions treated by regulation, stabilization and normalization of enterochromaffin cell functions, GI secretion, motility, afferent and efferent fiber activity and/or abdominal smooth muscle cell activity.

The term “gastro-esophageal reflux disease” and “GERD” as used herein means the incidence of, and the symptoms of, those conditions caused by the reflux of the stomach contents into the esophagus. This includes all forms/manifestations of GERD including, but not limited to, erosive and non-erosive GERD, heartburn and other symptoms associated with GERD.

The term “irritable bowel syndrome” and “IBS” as used herein means a disorder of function involving altered motility, sensitivity and secretion involving primarily the small intestine and large bowel associated with variable degrees of abdominal pain, bloating, constipation or diarrhea without overt bowel inflammation.

The term “dyspepsia” as used herein means a condition characterized by symptoms of epigastric pain, abdominal pain, bloating, early satiety, nausea, heartburn and vomiting as a primary gastrointestinal dysfunction or as a complication due, and not exclusive to disorders such as ulcer disease, appendicitis, gallbladder disturbances, or malnutrition.

The term “gastroparesis” as used herein means a paralysis of the stomach brought about by a motor abnormality in the stomach which is often manifested as delayed gastric emptying. This can also be a complication of diseases such as diabetes, progressive systemic sclerosis, anorexia nervosa, or myotonic dystrophy.

The term “constipation” as used herein means a condition characterized by infrequent and/or difficult evacuation of feces resulting from conditions such as altered GI motility, altered sensation or evacuation functions, altered secretion or reabsorption of electrolytes and water.

The term “diarrhea” as used herein means a condition characterized by frequent evacuations of feces often associated with large volumes and urge resulting from conditions such as altered GI motility, altered sensation and secretion or reabsorption of electrolytes and water.

The term “treat” or “treatment” encompasses the complete range of therapeutically positive effects associated with pharmaceutical medication including reduction of, alleviation of and relief from the symptoms or illness which affect the organism.

Preferably, the jointly therapeutically effective amounts of the active agents according to the combination of the present invention can be administered simultaneously or sequentially in any order, e.g. separately (combined pharmaceutical preparation) or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less gain of weight or less cardiovascular side effects).

The diseases, disorders or conditions related to diabetes, particularly type 2 diabetes mellitus, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of diabetic patients, e.g. reducing the risk of negative cardiovascular events, reducing risk of side effects, controlling increase of weight (in diabetic patients) or in patients suffering from an altered gastrointestinal motility, sensitivity and/or secretion disorder(s).

In view of reduced dose of the DPP-IV inhibitor or agent interacting with a 5-HT₃ or a 5-HT₄ receptors, used according to the present invention, there is a considerable safety profile of the combination making it suitable for first line therapy.

The pharmaceutical composition according to the present invention as described herein before and hereinafter may be used for simultaneous use or sequential use in any order, for separate use or as a fixed combination.

Method or use as described above, wherein the DPP-IV inhibitor and the agent(s) interacting with a 5-HT₃ receptor and/or an agent interacting with a 5-HT₄ receptor are administered in the form of a combination of the present invention such as a fixed combination or combined preparation or kit of part.

Combination, method or use as described herein, wherein the DPP-IV inhibitor is (S)-1-[(3-hydroxy-1-adamantyl)amino]acetyl-2-cyano-pyrrolidine and wherein the agent interacting with a 5-HT₄ receptor is preferably selected from the group consisting of tegaserod, cisapride, nor-cisapride, renzapride, zacopride, mosapride, prucalopride, buspirone, norcisapride or, in each case, a pharmaceutically acceptable salt thereof.

Combination, method or use as described above, wherein the DPP-IV inhibitor is (S)-1-{2-[5-cyanopyridin-2-yl)amino]ethyl-aminoacetyl)-2-cyano-pyrrolidine and wherein the agent interacting with a 5-HT₄ receptor is tegaserod or, in each case, a pharmaceutically acceptable salt thereof especially tegaserod hydrogen maleate.

Combination, method or use as described herein, wherein the DPP-IV inhibitor is (S)-1-[(3-hydroxy-1-adamantyl)amino]acetyl-2-cyano-pyrrolidine and wherein the agent interacting with a 5-HT₃ receptor is preferably selected from the group consisting of cilansetron, ramosetron, azasetron, ondansetron, dolasetron, ramosetron, granisetron, mirtazapine, indisetron, lerisetron, Ro-93777, YM-114, talipexole, N-3389, zacopride, cilansetron, E-3620, lintopride, KAE-393, itasetron, mosapride, dolasetron and tropisetron or, in each case, a pharmaceutically acceptable salt thereof.

According the invention, when the DPP-IV inhibitors, and the agent(s) interacting with a 5-HT₃ receptor and/or an agent interacting with a 5-HT₄ receptor are administered together, such administration can be sequential in time or simultaneous with, the simultaneous method being generally preferred. For sequential administration, the DPP-IV inhibitor, and the agent(s) interacting with a 5-HT₃ receptor and/or an agent interacting with a 5-HT₄ receptor can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the DPP-IV inhibitor, and the agent(s) interacting with a 5-HT₃ receptor and/or an agent interacting with a 5-HT₄ receptor are administered sequentially, the administration of each can be by the same method or by different methods.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;
(b) an amount of at least one therapeutic agent selected from components (i) to (ii), or, in each case, where appropriate, a pharmaceutically acceptable salt thereof in a second etc. unit dosage form; and
(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;
(b) an amount of at least one therapeutic agent selected from agent(s) interacting with a 5-HT₃ receptor and/or an agent interacting with a 5-HT₄ receptor or, in each case, where appropriate, a pharmaceutically acceptable salt thereof, in the form of two or three or more separate units of the components (a) to (b), especially for the prevention of, delay of progression of, treatment of a disease or condition according to the present invention.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g. 50 mg of LAF237.

The pharmaceutical composition according to the present invention as described hereinbefore may be used for simultaneous use or sequential use in any order, for separate use or as a fixed combination.

Thus according to a further embodiment, a DPP-IV inhibitor, is administered with an agent(s) interacting with a 5-HT₃ receptor and/or an agent interacting with a 5-HT₄ receptor preferably in the form of a fixed pharmaceutical composition comprising a pharmaceutically acceptable carrier, vehicle or diluent. Accordingly, a DPP-IV inhibitor of this invention, can be administered with an agent(s) interacting with a 5-HT₃ receptor and/or an agent interacting with a 5-HT₄ receptor as a fixed combination, in any conventional oral, parenteral or transdermal dosage form.

The doses of DPP-IV inhibitor of formula (I) to be administered to warm-blooded animals, for example human beings, of, for example, approximately 70 kg body weight, especially the doses effective in the inhibition of the DPP-IV enzyme, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

The dosage of (S)-1-[(3-hydroxy-1-adamantyl)amino]acetyl-2-cyano-pyrrolidine is preferably between 10 and 150 mg daily, most preferably between 25 and 150 mg, 25 and 100 mg or 25 and 50 mg or 50-100 mg daily. Preferred examples of daily oral dosage are 25, 30, 35, 45, 50, 55, 60, 80 or 100 mg. The application of the active ingredient may occur up to three times a day, preferably one or two times a day.

The preferred herein mentioned agent(s) interacting with a 5-HT₃ receptor and/or an agent interacting with a 5-HT₄ receptor, will be supplied in the form of suitable dosage unit form, for example, a capsule or tablet, and comprising a therapeutically effective amount, e.g. from about 2 to about 200 mg, as already described herein and in the prior art. The application of the active ingredient may occur up to three times a day, preferably one or two times a day. The same preferred dosage are selected for the fixed combinations.

Daily tegaserod dosages required in practicing the method of the present invention will vary depending upon, for example the mode of administration and the severity of the condition to be treated. An indicated daily dose is in the range of from about 1 to about 30 mg, e.g. from 2 to 24 mg or from 2 to 12 mg, of active agent for oral use, conveniently administered once or in divided dosages. Preferred galenic formulations used to deliver tegaserod are described in the International Patent Applications WO2003053432 especially in examples 1 to 3 and WO 00/10526, which are hereby incorporated by reference.

Corresponding doses may be taken, for example, in the morning, at mid-day or in the evening.

In a preferred aspect, the invention concerns a combination or use or a method as described herein, comprising or wherein the daily administration is;
i) between 25 and 150 mg or between 50 and 100 mg of vildagliptin, and
ii) between 1 and 30 mg or between 2 and 12 mg of tegaserod, or in any case, a pharmaceutically acceptable salt thereof.

The invention concerns a combination or use or a method as described herein, comprising or wherein the daily administration is;
50 mg of vildagliptin and 2 mg of tegaserod or in any case, a pharmaceutically acceptable salt thereof.
50 mg of vildagliptin and 6 mg of tegaserod or in any case, a pharmaceutically acceptable salt thereof.
50 mg of vildagliptin and 12 mg of tegaserod or in any case, a pharmaceutically acceptable salt thereof.
100 mg of vildagliptin and 2 mg of tegaserod or in any case, a pharmaceutically acceptable salt thereof.
100 mg of vildagliptin and 6 mg of tegaserod or in any case, a pharmaceutically acceptable salt thereof.
100 mg of vildagliptin and 2 mg of tegaserod or in any case, a pharmaceutically acceptable salt thereof.

Preferably, in case of free combinations, preferred are those dosages for launched products that have been approved and that have been marketed.

Especially preferred are low dose combinations.

To further illustrate the invention, but not by way of limitation, the following examples are provided.

### EXAMPLE 1

### Pharmacodynamic Effects of Tegaserod and LAF237 on Gastrointestinal and Colonic Motility

Animal preparation: Beagle dogs are used in these experiments. Under halothane anesthesia, four strain-gauge transducers constructed according to Pascaud et al. (Am. J. Physiol., 1978, 235: E532-E538) are sewn on the serosa of the antrum at 5 cm from the pylorus, the duodenum at 10 cm from the pylorus, the jejunum at 50 cm from the ligament of Treitz and the proximal colon at 10 cm from the ileo-colonic junction. Each transducer is sewn with its recording axis parallel to the transverse axis of the gut to measure the contractile force of the circular muscle layer. The free ends of the strain-gauge wires are drawn sub-cutaneously to emerge dorsally between the scapulas.

Recordings: Calibration of each strain-gauge is performed before implantation. Mechanical activity detected by the transducers is recorded. The motility index of the antrum, duodenum, jejunum and colon is determined according to the technique of Hachet et al. (J. Pharmacol. Meth. (1986) 16: 171-180). The calculated index of motility corresponds to the area between the baseline and the contractile curve during 30 min intervals.

Study design: The dogs are separated into groups. Each group receives one of the following regimens: 1) placebo, 2) tegaserod, 3) LAF237, 4) tegaserod plus LAF237. Compounds at different doses or placebo are administered p.o. to fasted dogs 30 min prior to a meal (water ad libitum). Intravenous infusions of compounds at different doses or placebo (vehicle) to fasted dogs started 30 min prior to a meal (water ad libitum). Gastrointestinal and colonic motility recordings start with the meal intake and are carried out for 6 hours duration in total.

Data analysis: Changes in motility index during the 6 hours after the meal associated with the different compounds/administrations are determined at the level of the antrum, duodenum, jejunum and colon.

The combination of tegaserod plus LAF237 can significantly increase gastrointestinal and colonic motility as compared to placebo and any of the compounds administered alone.

### EXAMPLE 2

### Effects of Tegaserod and LAF237 on Gastric and Colonic Sensitivity to Distension and on the Muscular Tone of the Gut Using Barostatic Distension

### 1. 1. Gastric Sensitivity and Tone

Groups of Wistar rats weighing 200-250 g are used. For surgery, the animals are premedicated with 0.3 ml of acepromazine (0.5 mg/kg) injected intraperitoneally (ip) and anesthetized with 0.3 ml of ketamine injected intraperitoneally.

Animals are positioned in dorsal decubitus and following a xypho-ombilical laparotomy, the stomach is fitted with a permanent balloon connected to a tube introduced in the upper part of the rumen at 1 cm of the gastro-esophageal junction on the great curvature. After closure of the abdomen, rats are positioned in ventral decubitus and one group of 3 stainless steel electrodes (1 m in length-270 μm in diameter) is implanted into the neck muscles using a technique described in Ruckebusch and Fioramonti, Gastroenterol. 68:1500-1508, 1975. The free ends of electrodes and the catheter of the balloon are exteriorized on the back of the neck and protected by a glass tube attached to the skin.

Gastric distension at constant pressure is performed with an electronic barostat (Hachet et al., Gastroenterol Clin Biol, 1993, 17, 347-351). Balloons (5.0-5.5 cm in length) are made with cistern free condoms and sutured to a polyethylene tube (1.0 and 1.8 mm inner and outer diameter respectively, 80 cm in length). The end of the tube is drilled for an easier emptying of the balloon.

Ten days after surgery, electromyographic recordings are performed with an electroencephalograph machine (Reega VIII, Alvar, Paris, France) at a paper speed of 2.4 cm/min. A short time constant of amplification is used to record selectively spike burst (0.03 s). The electromyographic activity is summed every 20 s by an integrator circuit and automatically plotted on a computer.

Under noxious gastric distension, the rat stretches its body and rises up the head and/or turns the head on the left and right sides to observe his flank. The neck muscles are contracted and an electromyographic signal is recorded. In addition, the barostat is connected to a potentiometric recorder for the permanent recording of intragastric pressure. The animals are separated into groups.

After a 30 min period of control recording, the animals receive one of the following regimens: 1) placebo, 2) tegaserod, 3) LAF237, 4) tegaserod+LAF237.

The protocol of gastric distension is started 30 min later.

Electromyographic activity of the neck muscles (EANM) is correlated with changes of posture and is proportional to pain induced by gastric distension. Values integrated every 20 s are summed up for consecutive 10 min. For each stage of distension, neck activity is determined with the following formula:

$$\frac{\begin{matrix}( E A N M at determined pressure ) - \\ ( E A N M in basal conditions )\end{matrix}}{E A N M in basal conditions} * 100$$

The pain threshold is determined as an increase >100% of the electrical activity of the neck muscles.

Gastric volume is determined on the potentiometric recorder as the maximal volume obtained for each stage of distension. Pain threshold and gastric volume are given as mean±SEM and values compared using Student's “t” test for unpaired values.

The pharmaceutical combination of tegaserod and LAF237 can significantly decrease the gastric pain associated with gastric distension and increases in gastric tone as compared to placebo and any of the compounds administered alone.

### 2. Colorectal Sensitivity and Tone

The influence of tegaserod and LAF237 on rectal or colonic tone and pain is done using barostat distension procedures by applying increasing pressure in a stair-case manner for consecutive periods of 5 min.; the volume is measured for each pressure giving an evaluation of the changes in tone.

Wistar rats weighing 220-250 g and housed individually are used. The animals are premedicated with 0.5 mg/kg of acepromazine injected intraperitoneally (IP) and anesthetized by intramuscular administration of 100 mg/kg of ketamine. They are prepared for electromyographic recordings using the technique described in Ruckebusch and Fioramonti, 1975. Pairs of nichrome wire electrodes (60 cm in length and 80 μm in diameter) are implanted in the striated muscle of the abdomen, 2 cm laterally from the white line. The free ends of electrodes are exteriorized on the back of the neck and protected by a plastic tube attached to the skin.

Electromyographic recordings (time constant: 0.03 sec) started 8 days after surgery. Bipolar recordings of myoelectric activity are performed with an electroencephalographic recorder during one hour starting 30 min before rectal distension.

In order to prevent recording artefacts due to movements during distension, rats are acclimated, 3 days before distension, to stay in tunnel of polypropylene in which distension and EMG recordings are performed. A balloon consisting of a condom (4 cm) is introduced into the rectum at 5 cm from the anus and fixed at the base of the tail. The balloon, connected to a barostat, is increasingly inflated with air at pressures of 15, 30, 45 and 60 mmHg. each pressure being applied during 5 min.

Groups of rats are submitted respectively to the barostatic distension protocol. Ten minutes before they are injected IP with 1) placebo, 2) tegaserod, 3) LAF237, 4) tegaserod+LAF237. Statistical analysis of the number of abdominal spike bursts occurring during each 5 min period are performed by Student's “t” test fair paired values comparisons after two way ANOVA. P<0.05 is considered statistically significant. Colorectal volumes are given as mean±SEM and values compared using Student's “t” test for unpaired values.

The pharmaceutical combination of tegaserod plus LAF237 can significantly decreases the rectal and colonic pain associated with rectal distension and increases colorectal tone as compared to placebo and any of the compounds administered alone.

### EXAMPLE 3

### Treatment of Non-Erosive GERD with the Combination of Tegaserod and LAF237

Patients selected for the study are patients with heartburn, the target symptom in patients with non-erosive GERD, as the predominant upper gastrointestinal symptom during the last three (3) months prior to entry into the study and with a history of episodes of heartburn occurring on at least 3 days/week. Patients with GERD and without endoscopic signs of erosive esophagitis are included in the study. Among other factors, patients who are treated with histamine H₂-receptor antagonists (H₂RAs) in prescription doses or PPls within one month prior to entry into the baseline phase of the study (Day-14) and patients who need continuous use of PPIs within three months prior to entry into the baseline phase of the study are excluded.

The study consists of a one-week screening period and a 2-week drug-free baseline period, followed by an 8-week, double-blind, placebo-controlled treatment period. During the screening period (Day-21 to Day-14), an endoscopy is performed to rule out the presence of erosive esophagitis. During baseline (Day-14 to Day 1), the patient's symptoms of GERD are documented in a daily diary. At the start of the period, medications for GERD such as H₂RAs, PPls, prokinetics and other disallowed medication are withdrawn and the patients are instructed not to change their diet or lifestyle during the trial. Patients are allowed to take Maalox tablets as a rescue medication for the control of their symptoms. Patients entering the double-blind period have episodes of heartburn on three (3) or more days the last week of the baseline period.

Patients are randomized in equal groups during the double-blind, placebo-controlled period of the study. This period of the study lasts eight (8) weeks and there are 12 treatment arms. The patients in each group receive one of the following regimens: 1) placebo, 2) tegaserod 0.4 mg/day, 3) tegaserod 1 mg/day, 4) tegaserod 4 mg/day, 5) LAF237 50 mg/day, 6) tegaserod 0.4 mg/day plus LAF237 50 mg/day, 7) tegaserod 1 mg/day plus LAF237 50 mg/day, 8) tegaserod 4 mg/day plus LAF237 50 mg/day, given orally bid for the 8 weeks. The administration as per the twelve (12) groups above is within thirty (30) minutes before meal time in the morning and in the evening. During the 8 weeks, patients continue to complete the daily diary and use only Maalox tablets as rescue medication for the control of their symptoms.

The combination of tegaserod with LAF237 can significantly reduce the episodes of heartburn occurring per week during the 8 week period of the double-blind, placebo-controlled period of the study as compared to any of placebo, tegaserod, and LAF237 alone.

The tegaserod combinations also reduces the other symptoms of GERD including, abdominal pain, bloating and regurgitation. Further, patients show a significant improvement in quality of life factors as compared to any of placebo, tegaserod, and LAF237 alone.

A pharmaceutical combination according to the invention, e.g. comprising a 5-HT₄ agonist or partial agonist, e.g. tegaserod, and a DPP-IV inhibitor, e.g. LAF237, may also be tested clinically, e.g. using a methodology as disclosed by Talley N J, et al., in Gastroenterol. Intl., 1993, 6(4), 189:211, or by Veldhuyzen van Zanten S J O et al., in Gut 1999, 45 (Suppl. II), 1169:1177. The dosage is preferably oral and administration may be preferably once or twice a day.

The above described examples can also be used to prove the efficacy of DPP-IV inhibitors alone for treating the diseases and conditions of the invention. The dosages and formulations described herein for the combination therapy can also be applied if DPP-IV is used as monotherapy.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth herein in full.

What is claimed is:

1. A pharmaceutical combination, comprising:
i) a DPP IV inhibitor or a pharmaceutically acceptable salt thereof, and
ii) at least one therapeutic agent selected from an agent interacting with a 5-HT₃ receptor and/or an agent interacting with 5-HT₄ receptor, or a pharmaceutically acceptable salt thereof.

2. The pharmaceutical combination according to claim 1 further comprising at least one additional pharmaceutically acceptable carrier.

3. The pharmaceutical combination according to claim 1, in the form of a combined preparation or a fixed combination.

4. (canceled)

5. A method for the prevention of, delay of progression of, treatment of diseases and disorders, comprising:
administering to a warm-blooded animal in need thereof a jointly effective amount of a combination of a DPP IV inhibitor or a pharmaceutically acceptable salt thereof with at least one therapeutic agent selected from an agent interacting with a 5-HT₃ receptor and/or an agent interacting with a 5-HT₄ receptor, or a pharmaceutically acceptable salt thereof;
and at least one additional pharmaceutically acceptable carrier.

6. The method according to claims 5, wherein the disease or disorder is selected from insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, ulcerative colitis, Crohn's disease, ulcers and the visceral pain associated therewith.

7. The pharmaceutical combination according to claim 1, wherein the DPP-IV inhibitor is selected from (S)-1-{2-[5-cyanopyridin-2-yl)amino]ethyl-aminoacetyl)-2-cyano-pyrrolidine, vildagliptin, MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, or in each case, a pharmaceutically acceptable salt thereof.

8. The pharmaceutical combination according to claim 1, wherein the DPP-IV inhibitor is vildagliptin or a pharmaceutically acceptable salt thereof.

9. The pharmaceutical combination according to claim 1, wherein the wherein the agent interacting with a 5-HT₄ receptor is selected from the group consisting of tegaserod, cisapride, nor-cisapride, renzapride, zacopride, mosapride, prucalopride, buspirone, and norcisapride or, in each case, a pharmaceutically acceptable salt thereof.

10. The pharmaceutical combination according to claim 1, wherein the agent interacting with a 5-HT₄ receptor is tegaserod or, in each case, a pharmaceutically acceptable salt thereof.

11. The pharmaceutical combination according to claim 1, wherein the agent interacting with a 5-HT₃ receptor is selected from the group consisting of cilansetron, ramosetron, azasetron, ondansetron, dolasetron, ramosetron, granisetron, mirtazapine, indisetron, lerisetron, Ro-93777, YM-114, talipexole, N-3389, zacopride, cilansetron, E-3620, lintopride, KAE-393, itasetron, mosapride; dolasetron and tropisetron or, in each case, a pharmaceutically acceptable salt thereof.

12. (canceled)

13. A method for the prevention of, delay of progression of, treatment of diseases and disorders selected from altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, ulcerative colitis, Crohn's disease, ulcers and the visceral pain associated therewith, comprising:
administering to a warm-blooded animal in need thereof an effective amount of a DPP IV inhibitor or a pharmaceutically acceptable salt thereof.

14-15. (canceled)

16. The pharmaceutical combination according to claim 8, wherein vildagliptin is administered in an amount between 25 and 150 mg daily.

17. The pharmaceutical combination according to claim 10, wherein tegaserod is administered in an amount between 1 and 30 mg daily.

18. The pharmaceutical combination according to claim 3, wherein the DPP IV inhibitor or a pharmaceutically acceptable salt thereof is vildagliptin, the agent interacting with 5-HT₄ receptor, or a pharmaceutically acceptable salt thereof is tegaserod, and
i) between 25 and 150 mg of vildagliptin, and
ii) between 1 and 30 mg of tegaserod,
or in any case, a pharmaceutically acceptable salt thereof.

19. The pharmaceutical combination according to claim 18, wherein,
i) 50 mg of vildagliptin, and
ii) tegaserod in an amount selected from the group consisting of 2 mg, 6 mg and 12 mg,
or in any case, a pharmaceutically acceptable salt thereof.

20. The pharmaceutical combination according to claim 18, comprising;
iii) 100 mg of vildagliptin, and
iv) tegaserod in an amount selected from the group consisting of 2 mg, 6 mg and 12 mg,
or in any case, a pharmaceutically acceptable salt thereof.

21. (canceled)

22. The pharmaceutical combination according to claim 10, wherein the agent interacting with a 5-HT₄ receptor is tegaserod hydrogen maleate.
